(19) United States
(12) Patent Application Publication
(10) Pub. No.: US 2009/0047330 A1
(43) Pub. Date: Feb. 19, 2009

(54) Oral fast dissolving films for erectile dysfunction bioactive agents

(76) Inventor: Ramesh Bangalore, Pleasanton, CA (US)

Correspondence Address: RAMESH BANGALORE, 5274 RIVERDALE CT, Pleasanton, CA, 94588, US

(21) Appl. No.: 12/228,702

(22) Filed: Oct. 9, 2008

Related U.S. Application Data
(60) Provisional application No. 60/965,047, filed on Aug. 17, 2007.
(60) Provisional application No. 60/964,950, filed on Aug. 17, 2007.
(60) Provisional application No. 60/965,022, filed on Aug. 17, 2007.

(51) Int. Cl. A61K 9/70; A61K 31/519; A61K 31/4985

(52) U.S. Cl. 424/443; 514/262.1; 514/250

(57) ABSTRACT

A novel edible polymer based film dosage form manufactured using natural, synthetic, semisynthetic, pharmaceutically acceptable polymers addressing the issues of swallowing difficulties (Dysphagia and Dynaphagia), of tablet or capsule dosage forms and handling and storage difficulties associated with liquid dosage forms, that also includes materials such as emulsifying agents, suspending agents, buffering agents, effervescence agents, colorants, flavorants, sweeteners and specified amounts of bioactive agents, for erectile dysfunction. A flexible film dosage form containing sildenafil citrate, tadalafil or Vardenafil is presented. The film system is enabled to be used in various applications such as oral, mucosal and external environments.

### CLAIM OF PROVISIONAL APPLICATION RIGHTS

This application claims the benefit of U.S. Provisional Patent Application Nos. 60/965,047; 60/964,950; 60/965,022 filed on Aug. 17, 2007.

### TECHNICAL FIELD OF INVENTION

The present invention is related to fast dissolving films containing an effective dose of erectile dysfunction active agents that are for immediate release.

### BACKGROUND OF INVENTION

It is common experience to the patient that swallowing of tablets and capsule may pose certain difficulty in dose administration. This physiological condition is known as “Dysphagia”, difficulty in swallowing or “Dynophagia”, painful swallowing. Such conditions are often experienced by elderly, patients with neck/head injuries or AIDS patients. It is also a common experience with pediatric patients who tend to be non compliant with solid oral dosage form administration. To address the issue of delivery of drugs to such patients several novel delivery systems such liquid dosage forms have been developed. However, handling the liquid systems and delivering liquids is always a challenge. Therefore, solid dosage forms with the convenience of liquids and the dose precision of solid dosage form is desirable. Additionally, dosage forms that do not need any liquids for administration exclusively are desirable for dose administration to dialysis patients who have the restriction on liquid consumption. This feature in dosage forms makes dose administration convenient even for the patients on go improving overall dose administration compliance.

In addition, the standard oral dosage forms, such as tablets, pills, caplets, and capsules, are designed for short residence time in the mouth. Absorption of the agent from these dosage forms occurs in the gastrointestinal (GI) tract only after the dosage form disintegration followed by dissolution of the agent in the gastric fluids. However, for some active agents, it is desirable to achieve absorption through the highly vascularized oral mucosal tissues in order to accelerate onset of the therapeutic effect.

Many active agents are poorly absorbed, even after they are dispersed in the stomach, because of low solubility or slow dissolution rate in the gastric fluids. Tablets may be formulated so as to be quick dissolving. These tablets are commonly placed on the tongue and disintegrate rapidly in the oral cavity. However, these dosage units are not fixed to a mucosal surface and may move around in the mouth. Consequently, they do not overcome a risk associated with choking or gagging that occurs with subjects having limited control of their swallowing reflexes. However, once placed in the mouth, these tablets dissolve rapidly in the saliva to provide a liquid formulation which is then swallowed. However, the disintegration/dispersion time for such tablet dosage forms may take sometimes more than one to few minutes. The quick dispersing/dissolving tablets may be formed from a particulate support matrix containing the therapeutic agent, where the particulate support matrix is a protein (U.S. Pat. Nos. 5,807,576, 5,635,210, 5,595,761). Alternatively, the tablet maybe formed from a laminate with several layers and an outer coating (JP 100535518). Tablets have also been manufactured from shear form matrices which are substantially amorphous sugar formed when crystalline sugar is subjected to heat and shear (WO 95/07194; WO 95/35293). Other methods of forming quick dissolving tablets include wet granulation methods (EP 0627 218) and dry granulation methods (EP 0124027A1) and by freeze-drying techniques (EP 0084705A2). Generally, quick dissolving tablets are formed using complex multi-step manufacturing processes. In addition, these tablets may have poor mechanical strength, are fragile and friable and have insufficient holding capacity for active ingredients (U.S. Pat. No. 5,720,974) and may be difficult to store and handle.

Many times therapeutic compounds provided as powders or granules and may be difficult to swallow and cause unpleasant sensations in the mouth. Furthermore, many quick dissolving tablets contain particulates (>25 microns) which leave a “gritty” and unpleasant taste in the mouth. In the elderly, powders may cause choking and discomfort associated with trapping of granules in dentures. Powders and granules are generally packaged in a sealed pouch which requires tearing before use. This causes problems for geriatric patients and those suffering from arthritis in the fingers as well as for children. Consequently, problems of spillage of the contents arise in this group of patients. Furthermore, these oral preparations should be taken with water which for certain patients are inconvenient and may cause reduced patient compliance.

Few of the alternatives to solid oral dosage forms addressing aforementioned disadvantages are liquids, syrups, emulsions or suspensions and are considered desirable for pediatric and geriatric patients who have problems in swallowing tablets. However, these dosage forms are often difficult to measure accurately and administer easily without the loss of dose due to spillage. Additionally, liquid formulations deteriorate rapidly upon exposure to heat or atmosphere and consequently have a relatively short shelf life while emulsion/suspension dosage forms may eventually settle and form cakes leading to the loss of dose uniformity. Furthermore, liquid formulations require a relatively large volume and are bulky to store.

In addition to solid and liquid dosage forms, rapidly dissolving buccal/oral delivery systems have been developed using methods including freeze drying. However, the freeze dried preparations are more expensive to manufacture as compared to tablets (U.S. Pat. No. 5,648,093). Furthermore, freeze dried preparations are brittle and fragile when handled and must be kept in dry conditions to avoid disintegration. Patent# WO 9820862 reports a film that is formed according to a method that does not utilize freeze drying and avoids problems described in the art such as rigidity of the films, delayed softening and poor solubility in the mouth (U.S. Pat. No. 4,876,092; EP 0200508; EPO 381194; CA-PS1-26331; DE 2449865.5; DE 3630603; EP 0452446 and EP 0219762). However, the film described in WO 9820862 relies on the use of at least two different non-ionic surfactants to achieve immediate wettability.

A delivery device that addresses the above limitations would represent a desirable improvement on existing delivery systems. In an effort to overcome the difficulties associated with self-administration of the traditional tablet dosage forms, orally disintegrating dosage forms, such as thin films for delivering a pharmacologically active agent via the oral cavity have been developed. The thin films are generally thin strip solid dosage forms incorporating a pharmacologically active agent, and which disintegrate in the oral cavity to release the active agent that is incorporated in the film. A wide variety of pharmacologically active agents have been incorporated into disintegrable thin film dosage forms.

Additionally, the thin film dosage forms fail to accommodate higher drug loading due to the limited thickness of the thin film in order to preserve the fast dispersing/dissolving characteristics of the films. Therefore, in the current invention the films can be made thicker to accommodate higher drug loading while keeping the fast dispersion/dissolution of the dosage form by incorporating perforations in the body of the film.

### Prior Art for Fast Dissolving Films:

It is quite a well known fact that pediatric and geriatric patients who have problems in swallowing tablets liquid, syrups or suspensions are alternatives to solid dosage forms and are considered desirable. However, the portability with relatively large volumes and stability upon exposure to heat or atmosphere are main disadvantages.

In addition to conventional oral dosage forms, rapidly dissolving buccal/oral delivery systems have been developed to deliver several therapeutic agents. Majority of these systems are freeze dried preparations which are more expensive to manufacture as compared to tablets (U.S. Pat. No. 5,648,093). In addition, freeze dried preparations are difficult to handle due to their physical properties such as brittleness and fragility and must be kept in dry conditions to avoid disintegration.

U.S. Pat. No. 6,531,114 reported methods and chewing gum formulations for delivering a sildenafil citrate. However, this method will have uncontrolled rate and extent of drug delivery that seriously varies the drug efficacy.

U.S. Pat. No. 6,803,031 describes delivery of erectile dysfunction drugs through an inhalation route. Aerosols used in this delivery system comprise particles of at least 5 percent by weight of an erectile dysfunction drug. However, safety of long term use of these drugs to lungs is yet to be proven along with variabilities associated with the delivery itself.

A dosage unit comprising a mucoadhesive film with an effective dose of a sexual dysfunction active agent is described in US patent 20030068378. However, performance of the dosage form is dependent on mucoadhesive agent and the state of oral mucosa at the time of administration.

U.S. Pat. No. 5,741,511 outlines a transdermal drug delivery device for treating erectile dysfunction which comprises a patch containing pharmaceutically active ingredient and being directly applied to the male glans penis and its support and the rings for constricting the base part of the penis to aid the erection. Although it is painless and safe to use, it is limited by the time it takes to be effective.

Any dosage form that addresses the above limitations would represent an improvement among available methods of existing delivery systems.

### SUMMARY OF INVENTION

Objective of the current invention is to provide a water soluble polymer based edible films for the oral delivery of Sildenafil citrate, or Tadalafil or Vardenafil HCl as active agents for erectile dysfunction.

Another objective of the invention is to provide Sildenafil citrate, or Tadalafil or Vardenafil HCl in the form of oral fast dissolving/dispersing film for immediate release and to use as needed basis.

Another objective of the invention is to provide a means for providing Sildenafil citrate, or Tadalafil or Vardenafil HCl in solution or dispersion form upon application on the tongue for further absorption in the GIT.

Another objective of the invention is to provide increased surface area of Sildenafil citrate, or Tadalafil or Vardenafil HCl for faster dissolution over other solid oral dosage forms for the enhanced dissolution/dispersion

Another objective of the invention is to provide an immediate release drug delivery system for drugs that are either partially or completely insoluble in commonly used solvents.

Another objective of the invention is to provide flexible usage of hydroxyl propyl β-cyclodextrins and randomly polymerized β-cyclodextrins to mask the taste of drugs that are delivered in the oral fast dissolving/dispersing films.

The general purpose of the present invention is to provide a new oral drug delivery system that has several advantages over and novel features that result in new oral drug delivery system which is not anticipated, rendered obvious or even implied by any of the prior art of oral cavity delivery system.

To attain this, the present invention generally consists of polymer, plasticizer, buffer agent, sweeteners and drug formed into a flexible film.

Generally the polymers include either water soluble polymers or pH dependent polymers or thermoplastic polymers or combination thereof along with suitable amounts of water soluble/water miscible/water insoluble plasticizers, buffering agents, sweeteners, emulsifying agents and suitable amounts of drug substance. The resultant combination of these polymers, plasticizers, buffering agents, sweeteners and drugs can be a solution, suspension or emulsion which can be spread to form films upon drying.

These systems can be made either in the presence of water and/or any suitable organic solvent. Such prepared films usually can contain drug levels in higher percentages or at same percentage as present in commonly known films on the market. Additionally, these films can be dissolved or dispersed in oral cavity at faster rates than traditional films due to the presence of perforations throughout the body of the strip.

The perforations can be made in the body of the film by several known methods such use of pins/punches or laser drills or mechanical drills or conventional methods. The holes can be of any shape and size, however, the film upon perforations still provide specific amounts of drug when ingested.

In addition, these films can incorporate a poorly soluble drug at higher concentrations up to 100 mgs in the form of micro or nano particles that increase the overall surface area for drug to enhance dissolution and or absorption.

In embodiments of the invention, the dosage unit may further include any of the following features: a dry film thickness in the range of 1-20 mils, more particularly less than 10 mils, tensile strength greater than 2500 psi, a modulus in the range of 25,000-350,000 psi, a disintegration time in a range from 1-300 seconds, a dissolution time in a range from 10-600 seconds, and a percentage elongation less than 10%.

Embodiments of the invention provide a delivery system for bioactive agents and other active agents that will fast disperse/dissolve and completely release their contents on the applied surface such as tongue. The release of the active agent occurs without mastication or the need for intake of water. However, the dosage form can be administered with aid of water if needed. Furthermore, embodiments of the invention further provide improvements that include: improved organoleptic properties (smell and taste), and texture and feel of dosage forms intended to be placed in the oral cavity; a dosage form which “melts” in the mouth and leaves a smooth pleasant after feel following dissolution; Depending on the optimal program for a specific application of the invention, the disintegration time and the dissolution time can be controlled within a prescribed range by adjustment of the formulation and the thickness of the film. In some cases, it is desirable for release of the active agent to occur after dissolution of the film. For these applications, the active agent may be encapsulated in a material with dissolution properties that are different from those of the hydrocolloid. Encapsulation of the active agent also may be utilized to achieve masking of taste for active agents that are bitter. In some cases, two or more different active agents may be included in the film. An example where multiple active agents frequently are administered is cold medications, which often contain several active agents.

“Coating mixture” is defined here and in the claims as a viscous and homogeneous mixture of film forming polymers, active agents and other additives in water or a solvent or a mixture thereof. The coating solution is treated according to the method of the invention to form a film.

“Thickness” is defined here and in the claims by measurements in mil (a mil=one thousandth of an inch) determined using appropriate thickness gauges.

Flavoring agents include the essential oils or water soluble extracts of menthol, wintergreen, peppermint, sweet mint, spearmint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, camomile, sugar, dextrose, lactose, mannitol, sucrose, xylitol, malitol, acesulfame potassium, talin, glycyrrhizin, sucralose, aspartame, saccharin, sodium saccharin, sodium cyclamate and honey.

Emulsifying agents include polyvinyl alcohol, sorbitan esters, cyclodextrins, benzyl benzoate, glyceryl monostearate, polyoxyethylene alkyl ethers, polyoxyethylene stearates, poloxamer, polyoxyethylene castor oil derivatives, hydrogenated vegetable oils, bile salts, polysorbates and ethanol. Plasticizers may include glycerin, sorbitol, propylene glycol, polyethylene glycol, triacetin, triethyl citrate (TEC), acetyl triethyl citrate (ATEC) and other citrate esters.

Water soluble inert fillers include mannitol, xylitol, sucrose, lactose, maltodextrin, dextran, dextrin, modified starches, dextrose, sorbitol, and dextrates.

Effervescence and buffering agents include acidulants and alkalizing agents including citric acid, fumaric acid, lactic acid, tartaric acid, malic acid, as well as sodium citrate, sodium bicarbonate and carbonate, sodium or potassium phosphate and magnesium oxide.

Coloring agents may include FD & C coloring agents, natural coloring agents, and natural juice concentrates, pigments and opacifying agents such as titanium oxide, silicon dioxide and zinc oxide.

Stabilizers and preservatives may include anti-oxidants, chelating agents, and enzyme inhibitors such as ascorbic acid, vitamin E, butylated hyroxyanisole (BHA), butylated hydroxytoluene (BHT), propyl gallate, dilauryl thiodipropionate, thiodipropionic acid, gum guaiac, citric acid, edetic acid and its salts, glutathione, anti-microbial agents sodium benzoate, parabens and derivatives, sorbic acid and its salts, propionic acids and its salts, sulfur dioxide and sulfites, acetic acid and acetates, nitrites and nitrates and others.

In embodiments of the invention, a film former concentration can be in the range of 5-99% of the dry weight of the films, more particularly greater than 5-10%. These films have dry tack and wet tack properties that improve ease of handling and use. The low dry tack properties of the film provide for a physically attractive and easily handled film that is neither fragile nor sticky and can be easily removed from packaging and placed on a mucosal surface. These properties render the films suitable for easy making, packaging, handling and application.

In the embodiment of invention, the film forming polymer can be a hydrocolloid, a water soluble polymer film forming material which are pharmaceutically acceptable polymers from the following categories including synthetic and natural polymer material. These can include polyanionic, polycationic and uncharged polymer species including Cellulose polymers (synthetic) such as Hydroxypropylmethyl cellulose (HPMC), Hydroxy propyl cellulose (HPC), Methyl Cellulose (MC), Carboxymethyl cellulose (CMC), Starches, and Cellulose polymers (natural) such as Acacia, Tragakanth, Carrageenan, Pullulan and Other water soluble polymers including Polystyrene sulfonates, Polyethylene oxides/Polyethylene glycols, Polyacrylic acids, Polybenzenesulfonic acids, Polyethylenimine, Poly diallyldimethyl ammonium chloride, Polyallylamine hydrochloride, Polyvinyl pyrrolidone (PVP) and Gelatin The water soluble polymeric materials also can be pectin and derivatives, guar gum, xanthan gum, gellan sodium salt, propyleneglycol alginate, starches (amylose, amylopectin), modified starches, hydroxyethyl starch, pullulan, carboxymethyl starch, gum ghatti, okra gum, karaya gum, dextrans, dextrins and maltodextrins, konjac, acemannan from aloe, locust bean gum, tara gum, quince seed gum, fenugreek seed gum, scleroglucan, gum arabic, psyllium seed gum, tamarind gum, oat gum, quince seed gum, carrageenans, scleraglucan, succinoglucan, larch arabinogalactan, flaxseed gum, chondroitin sulfates, hyaluronic acid, curdlan, chitosan, deacetylated konjac, and rhizobium gum.

Additionally, in embodiments of the invention, the water soluble hydrocolloid may be a polypeptide or protein exemplified by gelatins, albumins, milk proteins, soy protein, and whey proteins. The hydrocolloid may further be selected from a group of synthetic hydrocolloids exemplified by any of the following: polyethylene-imine, hydroxyethyl cellulose, sodium carboxymethyl cellulose, carboxymethyl cellulose, hydroxypropyl cellulose, hydroxypropyl methyl cellulose, methyl cellulose, ethyl cellulose, polyacrylic acids, low molecular weight polyacrylamides and their sodium salts (carbomers), polyvinylpyrrolidone, polyethylene glycols, polyethylene oxides, polyvinyl alcohols, pluronics, tetronics, and other block co-polymers, carboxyvinyl polymers, and colloidal silicon dioxide.

In the embodiment of invention the film forming material may also be a water insoluble polymer film forming material which are pharmaceutically acceptable polymers from the following categories including synthetic and natural polymer material such as Acrylic/methacrylic acid copolymers including pH dependent polymers such as acrylic acid/methacrylic acid copolymers (eg: Eudragit L and S polymers), pH independent polymers including acrylic/methacrylate copolymers (eg: Eudragit RL and RS polymers), and Carbomer polymers (eg: Carbopols 931 and 974 polymers and others).

Using above polymers by themselves or in combination with other polymers and suitable amounts of plasticizer content the films can be made using a Braive laboratory bar coater or a BASF lab coater or Elcometer or other industrial film coating systems such Mathis AG coating system onto a polyester sheet and dried at suitable temperature to a suitable relative humidity and suitable thickness which is then perforated using methods such as drilling, burning, punching, laser drilling and even by matrix forming. Methods for manufacturing the film dosage form of the invention include the solvent casting methods or alternatively extrusion methods involving blending ingredients to form a film using mechanical force and moderate heat. A preferred embodiment of the invention utilizes a hydroxypropyl methyl cellulose having a methoxy content of about 19-30% and hydroxypropyl content of 7-12% and a molecular weight of approximately 50,000-250,000 daltons.

In addition to hydrocolloids and the active agents, the films may contain any or all of the following ingredients: emulsifying agents, solubilizing agents, wetting agents, suspending agents, taste modifying agents, plasticizers, active agents, water soluble inert fillers, preservatives, buffering agents, effervescence agents, coloring agents, and stabilizers. In a preferred embodiment, the percentage dry weight concentration of at least single ingredients incorporated in a film in each of the following categories is as follows: emulsifying agent (0.1%-10%), plasticizer (0.5-20%), active agents (0.01-80%), taste modifying agents (0.1-10%), coloring agents (0.01-5%), water soluble inert fillers (0.5-50%), preservatives (0.01-10%), buffering agents (0.1-10%) and stabilizers (0.01-5%).

In an embodiment of the invention, the film forming polymer was dissolved in water or hydroalcoholic mixture. The bioactive agent was either dissolved or dispersed in water or hydroalcoholic mixture and added to the polymer solution under mild agitation. In addition to the active agent and the film forming polymer, any of the ingredients listed above may be added and dispersed or dissolved uniformly in to hydrocolloid solution. The volatile active ingredients and flavoring agents can be incorporated before or after film forming. This homogeneous mixture (coating solution) with a solid content of 5-80% and a viscosity of 300-25000 cps was then degassed under vacuum. This coating material was then coated on to the non-siliconized side of a polyester film at 5-50 mil or 0.01 mm to 5 mm wet film thickness, more preferably 10 microns to 2000 micron wet film thickness and dried in an hot air oven at 40-200 C. The dry film formed by this process is a glossy, stand alone, self supporting, non-tacky and flexible film. The dry film is then cut into a suitable shape and surface area for administration. The size of the film varies based on the dose to be delivered. Unlike other conventional dosage forms, the film dose form therefore provides the flexibility of accommodating dose at ease by changing the dimensions of the film. The sized films are then packaged into a single unit pack, multi-unit packages including blisters and dispensers.

Presented examples in the invention are mere illustrations of reducing the invention to the practice but not limited to these illustrations. Other means and methods to achieve films with perforations are possible to the skilled specialist therefore, should not be considered as limitations of the invention.

Various examples provided here show that how the properties of the films vary when different type or grade of film forming polymer and other components such as plasticizers, emulsifiers, suspending agents, flavorants, colorants, stabilizers and others. These and other details of invention will be described in connection with the accompanying drawings, which are furnished only by the way of illustration and not in limitation of the invention, and in which drawings:

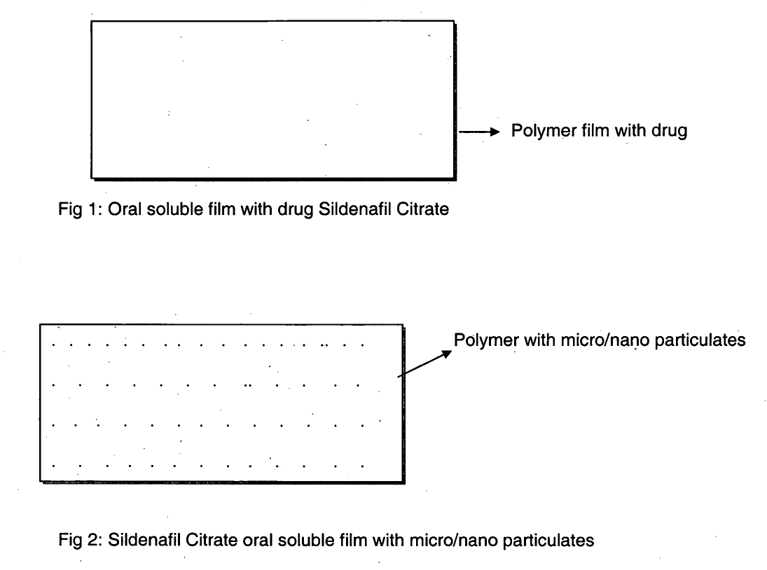

In its simplest embodiment, the current invention can be shown as presented in FIGS. 1-2. The following categories of polymers can be used in preparation of these films.

### EXAMPLES

In the embodiment of invention, the fast dispersing/dissolving perforated films are prepared using formulation compositions presented in Table 1.

The polymer based films are prepared as explained in the following examples using formulation composition presented in Table 1. The prepared polymer composition along with other ingredients including plasticizers, fillers, taste masking agents, disintegrants, colorants is then cast on to a backing membrane either using simple draw down blade or coated onto the moving roll of backing membrane and dried either in an oven or in the dynamic heating chamber.

The prepared films resulted in thickness range of 50 to 1000 microns based on the drug loading levels. Drugs from various pharmacological categories intended for oral application can be loaded into these perforated films. In the following example presented Sildenafil citrate is used in the preparation of fast dispersing/dissolving film dosage forms.

The following examples are set forth to further illustrate the oral films and methods of preparation. The below examples, however, should not be construed as limiting the present invention in any manner.

TABLE 1
Formulation compositions of fast dispersing/dissolving films

| | | Example | | | | Example | | | | Example | | | | Example | | | | Example | |
|---|---|---|---|---|---|---|---|---|---|---|---|---|---|---|---|---|---|---|---|
| S. No | Ingredient | -1 | | Example -2 | | -3 | | Example -4 | | -5 | | Example -6 | | -7 | | Example -8 | | -9 | |
| 1 | Methocel E 15 | 0.800 | g | 0.300 | g | 0.300 | g | | | | | 0.400 | g | 0.400 | g | | | | |
| 2 | Methocel E4 M | | | 0.100 | g | 0.100 | g | 0.100 | g | 1.000 | g | 0.150 | g | 0.150 | g | | | 0.100 | g |
| 3 | Methocel E 5 | | | | | | | 1.000 | g | | | | | | | 0.700 | g | 0.800 | g |
| 4 | Plasdone K 29-32 | 0.100 | g | 0.150 | g | 0.150 | g | | | | | | | | | | | | |
| 5 | Polyplasdone XL-10 | 0.100 | g | | | | | | | | | | | | | | | 0.150 | g |
| 6 | Sildenafil Citrate | 1.000 | g | 0.100 | g | 0.150 | g | 0.100 | g | 0.300 | g | 0.300 | g | 0.300 | g | 0.300 | g | 0.300 | g |
| 7 | HPBCD | 2.300 | g | | | | | | | | | | | | | | | | |
| 8 | Maltodextrin (M 180) | | | 1.500 | g | 1.500 | g | | | | | | | | | | | | |
| 9 | Instant purecote B 793 | | | | | | | 0.800 | g | 1.000 | g | | | | | 0.300 | g | 0.100 | g |
| 10 | Propylene glycol | 0.060 | g | 0.050 | g | 0.050 | g | 0.400 | g | 0.400 | g | | | 0.200 | g | | | | |
| 11 | Glycerol | 0.060 | g | 0.050 | g | 0.050 | g | | | | | 0.200 | g | 0.200 | g | | | 0.200 | g |
| 12 | Sorbitol (70% solution) | 0.2 | ml | 0.15 | ml | 0.200 | g | | | | | | | | | | | | |
| 13 | Triethyl citrate | | | | | | | | | | | | | | | | | | |
| 14 | Sucralose | 0.500 | g | 0.200 | g | 0.200 | g | | | | | 0.100 | g | 0.100 | g | | | 0.050 | g |
| 15 | Tween 80 | 0.1 | ml | 0.10 | ml | | | | | | | | | | | | | | |
| 16 | Menthol | 0.100 | g | | | | | | | | | | | | | | | 0.050 | g |
| 17 | MCC | | | | | | | | | | | | | | | | | 0.050 | g |
| 18 | Water | 8.00 | ml | 10.00 | ml | 10.00 | ml | 20.00 | ml | 15.00 | ml | 10.00 | ml | 10.00 | ml | | | 9.60 | ml |
| 19 | Ethanol | 12.00 | ml | | | | | | | | | | | 0.40 | ml | 15.00 | ml | 3.40 | ml |

### Example 1

0.8 g of Methocel E15, 0.1 g of Plasdone K29/300, 0.1 g of Polyplasdone XL10, 1.0 g of Sildenafil Citrate, 2.3 g of HPBCD (hydroxylpropyl β cyclodextrin), 0.06 g of Glycerol, 0.06 g of Propylene Gylcol, 0.2 ml of 70% Sorbitol solution, 0.5 g of Sucralose, 0.1 ml of Tween 80, 0.1 g of Menthol, 8 ml of water and 12 ml of Ethnol were added in the fashion presented in the preparation procedure followed by drying and perforating the resultant film. The formed films were uniform in appearance and able to dissolve rapidly.

### Example 2

0.3 g of Methocel E15, 0.1 g of Methocel E4M, 0.15 g of Plasdone K29/300, 0.1 g of Sildenafil Citrate, 1.5 g of Maltodextrin (M180), 0.05 g of Glycerol, 0.05 g of Propylene Gylcol, 0.15 ml of 70% Sorbitol solution, 0.2 g of Sucralose, 0.1 ml of Tween 80, 10 ml of water were added in the fashion presented in the preparation procedure followed by drying and perforating the resultant film. Good films were obtained with slight rough surface which dissolved instantly in the mouth. However, the drug loading in these films was 0.8 mg/sq.cm.

### Example 3

0.3 g of Methocel E15, 0.1 g of Methocel E4M, 0.15 g of Plasdone K29/300, 0.1 g of Sildenafil Citrate, 1.5 g of Maltodextrin (M180), 0.05 g of Glycerol, 0.05 g of Propylene Gylcol, 0.15 ml of 70% Sorbitol solution, 0.2 g of Sucralose and 10 ml of water were added in the fashion presented in the preparation procedure followed by drying and perforating the resultant film. In this formulation the surfactant Tween 80 was removed to obtain better films. Strong and slightly brittle dissolvable films which dissolved slightly slower than previous films in the mouth.

### Example 4

1.0 g of Methocel E5, 0.1 g of Methocel E4M, 0.8 g of Instant Purecote B793, 0.1 g of Sildenafil Citrate, 0.4 g of Propylene Gylcol and 20 ml of water were added in the fashion presented in the preparation procedure followed by drying and perforating the resultant film. The formed films were uniform, clear and transparent in appearance and able to dissolve rapidly. However, the drug loading was comparatively less in this formulation.

### Example 5

1.0 g of Methocel E5, 1.0 g of Instant Purecote B793, 0.3 g of Sildenafil Citrate, 0.4 g of Propylene Gylcol and 15 ml of water were added in the fashion presented in the preparation procedure followed by drying and perforating the resultant film. The formed films were at higher drug loading, uniform, clear and transparent in appearance and able to dissolve rapidly.

### Example 6

0.4 g of Methocel E15, 0.1 g of Methocel E4M, 0.3 g of Sildenafil Citrate, 0.2 g of Glycerol, 0.1 g of Sucralose, and 10 ml of water were added in the fashion presented in the preparation procedure followed by drying and perforating the resultant film. Good films were obtained with good mouth feel.

### Example 7

0.4 g of Methocel E15, 0.15 g of Methocel E4M, 0.3 g of Sildenafil Citrate, 0.2 g of Glycerol, 0.1 g of Sucralose, 10 ml of water and 0.4 ml of ethanol were added in the fashion presented in the preparation procedure followed by drying and perforating the resultant film. Good films were obtained with good mouth feel.

### Example 8

0.7 g of Methocel E5, 0.3 g of Instant Purecote B793, 0.3 g of Sildenafil Citrate, 0.2 g of Propylene Gylcol and 15 ml of water were added in the fashion presented in the preparation procedure followed by drying and perforating the resultant film. The formed films were at higher drug loading, uniform, clear, transparent and thick in appearance and able to dissolve rapidly over the unperforated films.

### Example 9

0.8 g of Methocel E5, 0.1 g of Methocel E4M, 0.15 g of Polyplasdone XL-10, 0.1 g of Instant Purecote B793, 0.3 g of Sildenafil Citrated, 0.2 g of Gycerol, 0.05 g of Menthol, 0.05 g of Microcrystalline cellulose, 9.6 ml of water and 3.4 ml of ethanol were added in the fashion presented in the preparation procedure followed by drying and perforating the resultant film. The formed films were uniform, clear and transparent in appearance and able to dissolve rapidly with higher drug loading.

The films from Example 9 were also cast to result into various dry film thicknesses as following:

### Trial 1

10×12 cm film was cast to get a film thickness of about 150 μm.

### Trial 2

10×12 cm film was cast to get a film thickness of about 225 μm.

### Trial 3

10×12 cm film was cast to get a film thickness of about 300 μm.

1. A oral fast dissolving or dispersing system for administration of drugs comprising a polymer based matrix system, suitable plasticizer, buffer agents, sweeteners, emulsifiers and ED class of drugs formed into film using various film forming systems.

2. The system in claim 1 where in the ED drug is Sildenafil Citrate.

3. The system in claim 1 where in the ED drug is Tadalafil

4. The system in claim 1 where in the ED drug is Vardenafil HCl

5. The system in claim 1 where the ED drugs are for immediate release and available as needed basis

6. The system in claim 1 for ED drugs are for immediate release by using appropriate embedded particulate systems

7. The system in claim 1 for ED drugs are for immediate release and for controlled release by using appropriate embedded particulate systems that potentially increase surface area, hence increases dissolution and permeability

8. The system in claim 1 for ED drugs can be dissolved or dispersed in oral cavity at faster rates than traditional films due to the presence of perforations throughout the body of the strip.

9. The system in claim 1 for ED drugs in combination with either hydroxyl propyl β-cyclodextrins and randomly polymerized β-cyclodextrins can mask the taste of drugs.

10. The system in claim 1 for ED drugs in combination with either hydroxyl propyl β-cyclodextrins or randomly polymerized β-cyclodextrins can solubilize agents that are otherwise insoluble in commonly used solvents.

11. The system in claim 1 for ED drugs are at dose levels from 1 to 50% of the film weight.

12. The system in claim 1 for ED drugs range from few micro grams to few hundred milligrams, more specifically from 10 micrograms to 500 milligrams.

13. The system in claim 1 for ED drugs comprises water soluble polymer/s wherein the water-soluble hydrocolloid is a polymer selected from the group consisting of a natural, semi-natural and synthetic biopolymer.

14. The system claim 1 for ED drugs, wherein the water-soluble hydrocolloid is selected from the group consisting of a polysaccharade and a polypeptide.

15. The system in claims 1 or ED drugs, wherein the water-soluble hydrocolloid comprises a hydroxypropylmethylcellulose polymer.

16. The system in claim 1 for ED drugs, wherein the hydroxypropylmethylcellulose polymer has a molecular weight of less than 250,000 Daltons, preferably having methoxy content of about 19-30% and hydroxypropyl content of 7-12%.

17. The system in claim 1 for ED drugs, wherein the film further comprises at least one of an emulsifier, a plasticizer, a taste modifying agent, a water soluble inert filler, a preservative, a coloring agent, a stabilizer, a disintegrant, effervescence agent and a buffering agent.

18. The system in claim 1 for ED drugs, wherein the film further comprises an emulsifier at a concentration in the range of 0 to 10% w/w of the unit dosage form.

19. The system in claim 1 for ED drugs also further comprises at least one of a sweetening agent in the concentration range of 0.1 to 10% w/w, a flavoring agent in the concentration range of 0.1 to 5% w/w, a taste masking agent in the concentration range of 0.01 to 2% w/w and a coloring agent of 0.01 to 4% w/w of the unit dosage form.

20. The system in claim 1 for ED drugs, wherein the film further comprises a water soluble inert filler present at a concentration in the range of 0 to 50% w/w of the unit dosage form.

21. The system in claim 1 for ED drugs, wherein the film further comprises a preservative present at a concentration in the range of 0.01 to 10% w/w of the unit dosage form.

22. The system in claim 1 for ED drugs, wherein the active agent is present at a concentration in the range of 0.01 to 80% w/w of the unit dosage form.

23. The system in claim 1 for ED drugs, in addition to hydrocolloids and the active agents, may contain any or all of the following ingredients: emulsifying agents, solubilizing agents, wetting agents, suspending agents, taste modifying agents, plasticizers, active agents, water soluble inert fillers, preservatives, buffering agents, effervescence agents, coloring agents, and stabilizers. In a preferred unit dosage form, the percentage dry weight concentration of at least single ingredients incorporated in a film in each of the following categories is as follows: emulsifying agent (0.1%-10%), plasticizer (0.5-20%), active agents (0.01-80%), taste modifying agents (0.1-10%), coloring agents (0.01-5%), water soluble inert fillers (0.5-50%), preservatives (0.01-10%), buffering agents (0.1-10%) and stabilizers (0.01-5%).

24. The system in claim 1 for ED drugs, wherein the sexual dysfunction active agent is sildenafil citrate.

25. The system in claim 1 for ED drugs, wherein the film has a dry film thickness in the range of 0.050 to 20 mil.

26. The system in claim 1 for ED drugs, wherein the film has a dry film thickness of less than 10 mils.

27. The system in claims 1 for ED drugs, wherein the film exhibits a dissolution time in the range of 1 to 600 seconds upon application.

28. The system in claim 1 for ED drugs, wherein the film exhibits a dispersion/dissolution time in the range of 1 to 300 seconds upon application to mucosal surface or skin.

29. The system in claim 1 for ED drugs, wherein the bioactive agent is encapsulated within a polymer, wherein the polymer is chemically or physically similar or distinct from the hydrocolloid to result in controlled dissolution of bioactive agent in relation to the film.

30. The system in claim 1 for ED drugs wherein the films are made with pH dependent polymers resulting in faster disintegration of the films.
